(19) United States
(12) Patent Application Publication
(10) Pub. No.: US 2022/0313894 A1
(43) Pub. Date: Oct. 6, 2022

(54) LIQUID MEDIUM FOR BUBBLE FORMATION DURING LASER LITHOTRIPSY

(71) Applicant: LUMENIS LTD., Yokneam (IL)

(72) Inventors: OMER PELED, HAIFA (IL); ARKADY KHACHATUROV, HAIFA (IL)

(73) Assignee: LUMENIS LTD., Yokneam (IL)

(21) Appl. No.: 17/710,351

(22) Filed: Mar. 31, 2022

Related U.S. Application Data
(60) Provisional application No. 63/170,455, filed on Apr. 3, 2021.

(51) Int. Cl. A61M 3/02; A61B 18/26

(52) U.S. Cl. CPC A61M 3/0254; A61B 18/26; A61B 2018/263; A61B 2018/00577

(57) ABSTRACT

The present disclosure provides medical devices and techniques for enhancing formation of vapor bubbles by laser energy in a liquid medium. In particular, the present disclosure is directed at a liquid medium with a tendency to develop and/or sustain a gaseous state in the presence of laser energy and a system to both irrigate a treatment site with the liquid medium and irradiate a target in the liquid medium.

### CROSS-REFERENCE TO RELATED APPLICATION

The present application claims the benefit of and priority to U.S. Provisional Patent Application Ser. No. 63/170,455 filed Apr. 3, 2021, entitled “SALINE”, the disclosure of which is incorporated herein by reference.

### BACKGROUND

Introduction of lasers into the medical field and the development of fiber optic technologies that use lasers has opened numerous applications in treatments, diagnostics, therapies, and the like. Such applications range from invasive and non-invasive treatments to endoscopic surgeries and image diagnostics. For instance, in urinary stone treatment, the stones are required to be fragmented into smaller pieces. A technology known as laser lithotripsy may be used for such fragmenting processes, wherein for small to medium sized urinary stones, a rigid or flexible ureteroscope is placed through the urinary tract for illumination and imaging. Simultaneously, an optical fiber is inserted through a working channel of the ureteroscope, to a target location (e.g., to the location where the stone is present in the bladder, ureter, or kidney). The laser is then activated to fragment the stone into smaller pieces or to dust it. In another instance, a laser and optic fiber technology is used in ablation treatments. During an ablation treatment, laser light is delivered to the tissue to vaporize the tissue or to induce damage within the tissue. Such ablation treatments may be used for treating various clinical conditions, such as Benign Prostate Hyperplasia (BPH), or cancers, such as prostate cancer.

The target to be treated (e.g., the tissue, the stone, etc.) is often immersed in a liquid medium, primarily consisting of water (e.g., saline, urine, etc.). It is to be appreciated that laser light is highly absorbed in water. When laser light is absorbed in the liquid medium, a vapor bubble is created. The vapor bubble expands outwardly towards the target. Once the bubble reaches the target, the laser light can pass through the vapor to the target with very little attenuation as the laser light is significantly less absorbed in the steam vapor than in the liquid medium.

In some applications, a first laser pulse is initiated to create the vapor bubble followed by a second laser pulse to deliver a therapeutic amount of laser energy to the target through the vapor bubble. In some other applications, multiple optical fibers can be introduced to the treatment site while laser energy from the first optical fiber can be initiated to create the vapor bubble and laser energy from the second optical fiber can be initiated to deliver a therapeutic amount of laser energy to the target through the vapor bubble.

### BRIEF SUMMARY

The present disclosure provides medical devices and techniques for enhancing formation of vapor bubbles by laser energy in a liquid medium. In particular, the present disclosure is directed at a liquid medium with a tendency to develop and/or sustain a gaseous state in the presence of laser energy.

In some embodiments, a method of treating a target, includes irrigating a treatment site with a bubble activation fluid, the bubble activation fluid comprising a liquid base and a compound dissolved in the liquid base. The method additionally includes delivering, via an optical fiber coupled to a laser source, laser light, wherein the laser light causes the liquid base of the bubble activation fluid to vaporize and the vapors to be trapped by the dissolved compounds to form a bubble in the treatment site, and wherein the laser light is transmitted through the bubble to be incident on a target.

In further embodiments, the method includes wherein the liquid base includes distilled water and the compound includes sodium chloride and wherein the bubble activation fluid is carbonated.

In further embodiments, the method includes wherein the liquid base includes distilled water and the compound includes a surfactant, a biocompatible surfactant, a detergent, or a foaming agent.

In further embodiments, the method includes wherein the liquid base includes distilled water and the compound includes a hydrophilic chain.

In further embodiments, the method includes wherein the liquid base includes distilled water and the compound includes a an organic amphiphilic

In further embodiments, the method includes wherein the dissolved compounds form micelle structures to trap portions of the vaporized liquid base to reduce a surface tension of the bubble.

In further embodiments, the method includes wherein the bubble activation fluid has a lower boiling point that saline solution and wherein the saline solution includes 9 grams of sodium chloride dissolved in 1 liter of distilled water.

In some embodiments, a system includes a laser source arranged to deliver laser light to a treatment site via an optical fiber, a fluid reservoir comprising a bubble activation fluid, the bubble activation fluid comprising a liquid base and a compound dissolved in the liquid base, and a pump coupled to the fluid reservoir, the pump rearranged to irrigate the treatment site with the bubble activation fluid; and wherein the laser light causes the liquid base of the bubble activation fluid to vaporize and the vapors to be trapped by the dissolved compounds to form a bubble in the treatment site and wherein the laser light is transmitted through the bubble to be incident on a target.

In further embodiments, the system includes wherein the liquid base includes distilled water and the compound includes sodium chloride and wherein the bubble activation fluid is carbonated.

In further embodiments, the system includes wherein the liquid base includes distilled water and the compound includes a surfactant, a biocompatible surfactant, a detergent, or a foaming agent.

In further embodiments, the system includes wherein the liquid base includes distilled water and the compound includes a hydrophilic chain.

In further embodiments, the system includes wherein the liquid base includes distilled water and the compound includes a an organic amphiphilic.

In further embodiments, the system includes wherein the dissolved compounds form micelle structures to trap portions of the vaporized liquid base to reduce a surface tension of the bubble.

In further embodiments, the system includes wherein the bubble activation fluid has a lower boiling point that saline solution, wherein the saline solution includes 9 grams of sodium chloride dissolved in 1 liter of distilled water.

In further embodiments, the system includes the optical fiber.

In further embodiments, the system includes wherein the laser source includes a Thulium fiber laser (TFL) or a Holmium:YAG laser.

In further embodiments, the system includes the optical fiber and wherein the laser source includes a Thulium fiber laser (TFL) or a Holmium:YAG laser.

In some embodiments, a fluid includes a liquid base and a compound dissolved in the liquid base, wherein the liquid base is arranged to vaporize responsive to laser light, and wherein the dissolved compounds are arranged to trap portions of the vaporized base to form a bubble in the liquid base.

In further embodiments, the fluid includes wherein the liquid base includes distilled water and the compound includes sodium chloride and wherein the bubble activation fluid is carbonated.

In further embodiments, the fluid includes wherein the liquid base includes distilled water and the compound includes a surfactant, a biocompatible surfactant, a detergent, or a foaming agent.

In further embodiments, the fluid includes wherein the bubble activation fluid has a lower boiling point that saline solution, wherein the saline solution includes 9 grams of sodium chloride dissolved in 1 liter of distilled water.

These and other embodiments of the present disclosure will be readily apparent from the following detailed description.

### BRIEF DESCRIPTION OF THE SEVERAL VIEWS OF THE DRAWINGS

Non-limiting embodiments of the present disclosure are described by way of example with reference to the accompanying figures, which are schematic and not intended to be drawn to scale. It is to be appreciated that various figures included in this disclosure may omit some components, illustrate portions of some components, and/or present some components as transparent to facilitate illustration and description of components that may otherwise appear hidden. For purposes of clarity, not every component is labelled in every figure, nor is every component of each embodiment shown where illustration is not necessary to allow those of ordinary skill in the art to understand the disclosure. In the figures:

FIG. 1 illustrates a system in accordance with at least one embodiment of the present disclosure.

FIG. 2 illustrates a treatment site in accordance with at least one embodiment of the present disclosure.

FIG. 3 illustrates a micelle formed by dissolved compounds of a liquid medium in accordance with at least one embodiment of the present disclosure.

FIG. 4 illustrates a technique in accordance with at least one embodiment of the present disclosure.

FIG. 5 illustrates a system in accordance with at least one embodiment of the present disclosure.

### DETAILED DESCRIPTION

The foregoing has broadly outlined some features and technical advantages of the present disclosure, such that the following detailed description of the disclosure may be better understood. It is to be appreciated by those skilled in the art that the embodiments disclosed may be readily utilized as a basis for modifying or designing other structures for carrying out the same purposes of the present disclosure. For instance, components and features disclosed hereby may be selectively combined without departing from the scope of this disclosure. The novel features of the disclosure, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the claims. Further, although various embodiments may be described with respect to laser lithotripsy or laser ablation treatments, reference to these conditions should not be construed as limiting the possible applications of the disclosed aspects.

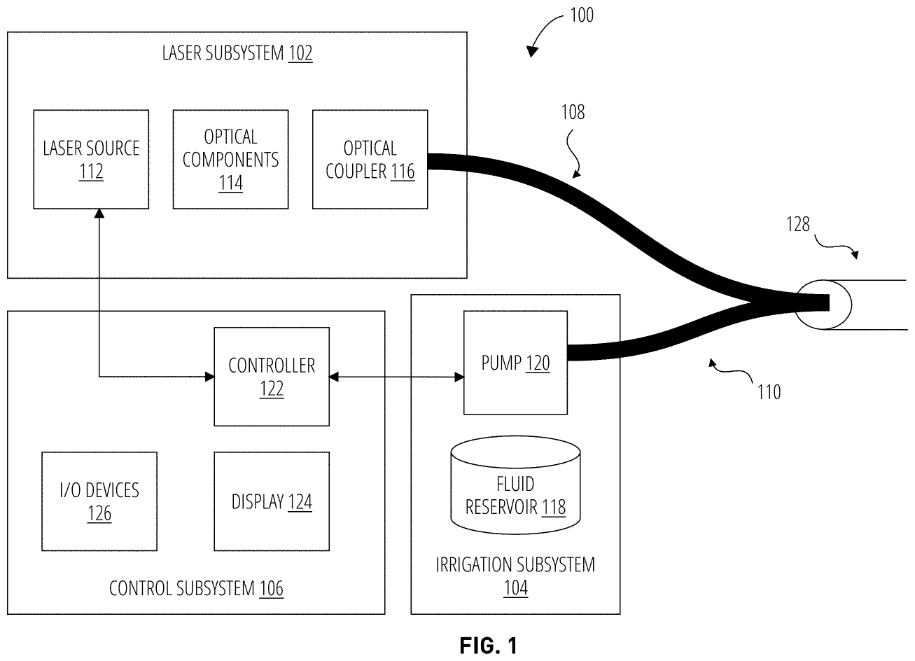

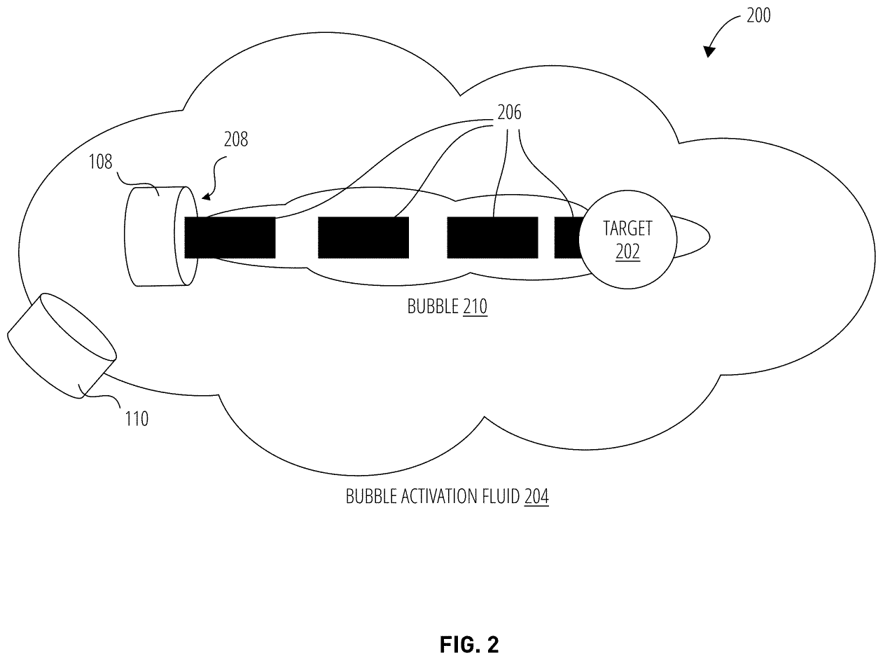

FIG. 1 illustrates a laser energy delivery system 100 in accordance with non-limiting example(s) of the present disclosure. The laser energy delivery system 100 can be used for a variety of procedures, such as, laser lithotripsy, laser ablation, or the like. The laser energy delivery system 100 includes a laser subsystem 102, an irrigation subsystem 104, a control subsystem 106, a fiber optic cable 108, and an irrigation tube 110. In general, the irrigation subsystem 104 is arranged to irrigate a treatment site (FIG. 2) with a liquid medium via the irrigation tube 110 while the laser subsystem 102 is arranged to generate laser light to be delivered to the treatment site via the fiber optic cable 108, where the liquid medium is configured to change from a liquid state to a gaseous state in the presence of the laser light to develop and/or sustain a vapor bubble through which the laser light can be transmitted from a distal end of the fiber optic cable 108 to a target.

The laser subsystem 102 includes a laser source 112, optical components 114, and optical coupler 116. The laser source 112 may comprise a laser arranged to generate a treatment beam. For example, the laser source 112 can be a Thulium fiber laser (TFL) source, a Holmium:YAG (Ho:YAG) laser source, or another type of laser source. The optical components 114 can include any of a variety of optical components (e.g., polarizers, beam splitters, beam combiners, light detectors, filters, wavelength division multiplexers, collimators, circulators, lenses, etc.) arranged to couple light emitted from laser source 112 with optical coupler 116 and ultimately fiber optic cable 108. The optical coupler 116 can be arranged to provide an optical coupling between a proximal end of fiber optic cable 108 and the laser subsystem 102 such that light emitted from the laser source 112 may be transmitted through the fiber optic cable 108 and emitted by the distal end of the fiber optic cable 108.

The irrigation subsystem 104 includes fluid reservoir 118 and pump 120. The fluid reservoir 118 is arranged to store a bubble activation fluid 204 while pump 120 is arranged to irrigate a treatment site with the bubble activation fluid 204 from fluid reservoir 118 via the irrigation tube 110.

The control subsystem 106 includes a controller 122, a display 124, and an input and/or output (I/O) devices 126. The controllers 122 can include circuitry such as an application specific integrated circuit (ASIC). As another example, the controller 122 can include a processor and memory storing instructions executable by the processor which when executed cause the laser energy delivery system 100 to implement the operations described herein. For example, the controller 122 can be operationally (e.g., communicatively, electrically, or the like) coupled to pump 120 and arranged to send control signals to pump 120 to cause the pump 120 to irrigate a treatment site with bubble activation fluid 204 from fluid reservoir 118. Similarly, the controller 122 can be operationally (e.g., communicatively, electrically, or the like) coupled to laser source 112 and arranged to send control signals to laser source 112 to cause laser source 112 to deliver laser energy to the treatment site while the treatment site is irrigated with bubble activation fluid 204 to such that vapor bubbles or a gaseous pathways are formed in the bubble activation fluid 204.

The display 124 can include any of a variety of displays (e.g., LCD displays, LEDs, or the like) arranged to provide a visual indication (e.g., graphical, a graphical user interface, graphical elements, light sequences, or the like) to communicate information to a user of the laser energy delivery system 100. The I/O devices 126 can include any of a variety of devices (e.g., mouse, keyboard, joystick, button, pedal, speaker, or the like) arranged to receiving input from a user or provide output to a user. With the display 124 and the I/O devices 126 a user can interact with (e.g., provide inputs to or received output from) the laser energy delivery system 100.

The fiber optic cable 108 and irrigation tube 110 can be introduced to the treatment site via a scope lumen 128, which can be for example, a lumen in a ureteroscope, an endoscope, or the like.

FIG. 2 illustrates a treatment site 200 including a target 202. The treatment site 200 can be a location within a body (e.g., a human body, an animal body, or the like) where the target 202 is located. As a specific example, the treatment site 200 can be a location inside a kidney or bladder while the target 202 is a kidney stone. As described above, the irrigation tube 110 can be introduced into the treatment site 200 via scope lumen 128 and the treatment site 200 irrigated with the bubble activation fluid 204 by the irrigation tube 110. Likewise, the fiber optic cable 108 can be introduced into the treatment site 200 via the scope lumen 128 (or another lumen) while laser pulses 206 are generated by the laser source 112 and emitted from the distal end 208 of the fiber optic cable 108. The laser pulses 206 interact with the bubble activation fluid 204 to form a bubble 210, or a series of bubbles (not shown) between the distal end 208 and the target 202 such that laser energy (e.g., the laser pulses 206, or the like) from the laser source 112 can be incident on the target 202.

Bubble activation fluid 204 can be water comprising one or more dissolved elements, chemicals, or compounds. With some examples, bubble activation fluid 204 can be distilled water comprising dissolved elements, chemicals, or compounds that reduce the boiling temperature such that the boiling temperature of the bubble activation fluid 204 is less than the boiling temperature of distilled water. With further examples, bubble activation fluid 204 can be distilled water comprising dissolved elements, chemicals, or compounds that reduce the boiling temperature and the chemical potential such that the boiling temperature and chemical potential of the bubble activation fluid 204 is less than the boiling temperature and chemical potential of distilled water. In a further example, the bubble activation fluid 204 can be distilled water comprising dissolved elements, chemicals, or compounds that reduce the boiling temperature, vapor pressure, chemical potential, and/or surface tension such that the boiling temperature, vapor pressure, chemical potential, and/or surface tension of the bubble activation fluid 204 is less than that of distilled water.

Conventionally, saline solution is often used as irrigation fluid. Saline contains sodium chloride (NaCl) dissolved in water. Typically, saline is 9 grams of NaCl per 1 liter of water. Saline is an isotonic or “normal” solution as it has a similar concentration to other bodily fluids. Saline is also an electrolyte solution, which is known to cause freezing point depression and boiling point elevation. Conversely, the present disclosure provides bubble activation fluid 204 which reduces boiling temperature.

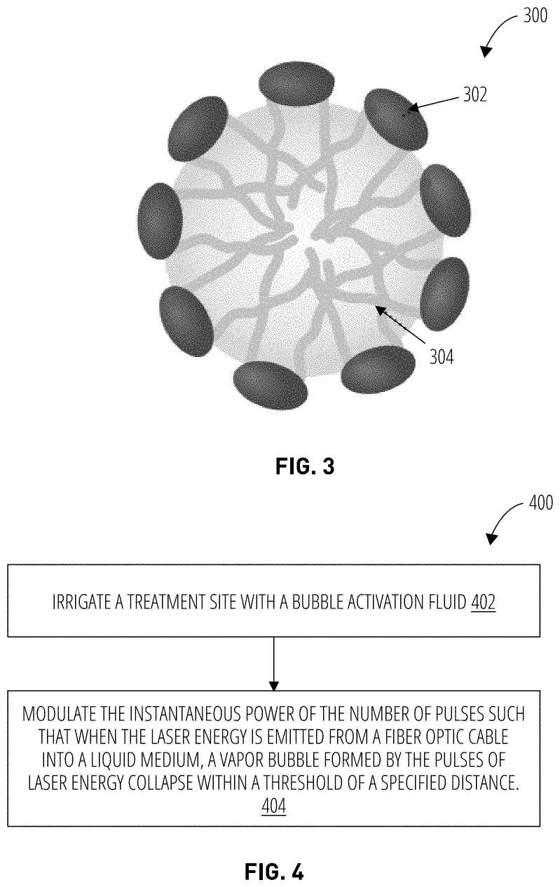

With some embodiments, the bubble activation fluid 204 can surfactants, detergents, and/or foaming agents dissolved in distilled water. As a specific example, bubble activation fluid 204 can be a biocompatible surfactant (e.g., a saponin, a saponin including carboxylic acid, or the like) dissolved in distilled water. As such, bubble activation fluid 204 can form micelle-like aggregates that act as bubble nucleation seeds (FIG. 3). As another specific example, bubble activation fluid 204 can be a hydrophilic chain (e.g., a sugar chain) having 1, 2, 3 (etc.) sugars. As another specific example, the bubble activation fluid 204 can be an organic amphiphilic compound. As such, bubble activation fluid 204 can have a reduced surface tension such that the bubble 210 may form with less energy or be maintained with less energy than a conventional bubble in a conventional liquid medium.

In some embodiments, the bubble activation fluid 204 can be gassed, bubbled, or carbonated saline (e.g., micro or macro) such that bubble nucleation sites are created in the bubble activation fluid 204. For example, the bubble activation fluid 204 can be carbonated saline where the NaCl in the saline forms micelles (FIG. 3). FIG. 3 illustrates a micelle 300. With some examples, bubble activation fluid 204 can include molecules dissolved in a liquid base (e.g., distilled water) that form micelles 300 in the presence of laser pulses 206 which acts to trap the water vapor thereby reducing the surface tension of the bubble.

As depicted, micelles 300 include a hydrophilic head 302 and hydrophobic tail 304. The hydrophobic tails 304 of several molecules assemble into core structure with the hydrophilic heads 302 forming an exterior of the micelles 300 and the tails intertwining in the interior of the micelles 300 in a manner that traps water vapor.

FIG. 4 illustrates a method 400 for treating a target with laser light. Method 400 can be representative of some or all of the operations that may be executed by one or more components, devices, or systems described herein, such as one or more portions of laser energy delivery system 100. As depicted, method 400 may begin at block 402. At block 402 “irrigate a treatment site with a bubble activation fluid” a treatment site is irrigated with a bubble activation fluid. For example, pump 120 may irrigate treatment site 200 with bubble activation fluid 204 from fluid reservoir 118.

Continuing to block 404 “activate a laser source to deliver laser light to the treatment site to vaporize portions of the bubble activation fluid to form micelles in the bubble activation fluid to trap vaporized particles of the bubble activation fluid” a laser source is activated to deliver laser light to the treatment site to vaporize portions of the bubble activation fluid such that micelles are formed in the bubble activation fluid to trap vaporized particles of the bubble activation fluid. For example, laser source 112 can be activated to deliver laser pulses 206 into bubble activation fluid 204 via fiber optic cable 108 to vaporize portions of bubble activation fluid 204 and form micelles 300 with molecules in bubble activation fluid 204 that trap the vaporized particles of bubble activation fluid 204.

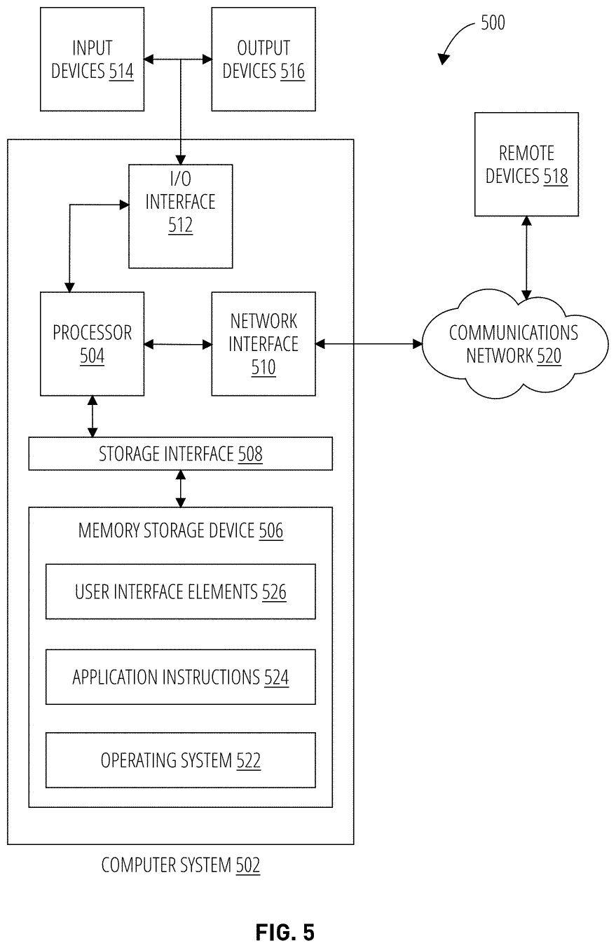

FIG. 5 is a block diagram of a computing environment 500 including a computer system 502 for implementing embodiments consistent with the present disclosure. In some embodiments, the computing environment 500, or portion thereof (e.g., the computer system 502) may comprise or be comprised in a laser system (e.g., the controller 122 of the laser energy delivery system 100 can embody portions of the computing environment 500). Accordingly, in various embodiments, computer system 502 may be used to control simultaneous irrigation of a treatment site with bubble activation fluid 204 and irradiation of the treatment site with laser light to form a bubble 210.

The computer system 502 may include a central processing unit (“CPU” or “processor”) 504. The processor 504 may include at least one data processor for executing instructions and/or program components for executing user or system-generated processes. A user may include a person, a person using a device such as those included in this disclosure, or another device. The processor 504 may include specialized processing units such as integrated system (bus) controllers, memory management control units, floating point units, graphics processing units, neural processing units, digital signal processing units, etc. The processor 504 may be disposed in communication with input devices 514 and output devices 516 via I/O interface 512. The I/O interface 512 may employ communication protocols/methods such as, without limitation, audio, analog, digital, stereo, IEEE-1394, serial bus, Universal Serial Bus (USB), infrared, PS/2, BNC, coaxial, component, composite, Digital Visual Interface (DVI), high-definition multimedia interface (HDMI), Radio Frequency (RF) antennas, S-Video, Video Graphics Array (VGA), IEEE 802.n/b/g/n/x, Bluetooth, cellular (e.g., Code-Division Multiple Access (CDMA), High-Speed Packet Access (HSPA+), Global System For Mobile Communications (GSM), Long-Term Evolution (LTE), WiMax, or the like), etc.

Using the I/O interface 512, computer system 502 may communicate with input devices 514 and output devices 516. In some embodiments, the processor 504 may be disposed in communication with a communications network 520 via a network interface 510. In various embodiments, the communications network 520 may be utilized to communicate with a remote memory storage device 506, such as for accessing look-up tables, performing updates, or utilizing external resources. The network interface 510 may communicate with the communications network 520. The network interface 510 may employ connection protocols including, without limitation, direct connect, Ethernet (e.g., twisted pair 10/100/1000 Base T), Transmission Control Protocol/Internet Protocol (TCP/IP), token ring, IEEE 802.11a/b/g/n/x, etc.

The communications network 520 can be implemented as one of the different types of networks, such as intranet or Local Area Network (LAN), Closed Area Network (CAN) and such. The communications network 826 may either be a dedicated network or a shared network, which represents an association of the different types of networks that use a variety of protocols, for example, Hypertext Transfer Protocol (HTTP), CAN Protocol, Transmission Control Protocol/Internet Protocol (TCP/IP), Wireless Application Protocol (WAP), etc., to communicate with each other. Further, the communications network 520 may include a variety of network devices, including routers, bridges, servers, computing devices, storage devices, etcetera. In some embodiments, the processor 504 may be disposed in communication with a memory storage device 506 via a storage interface 508. The storage interface 508 may connect to memory storage device 506 including, without limitation, memory drives, removable disc drives, etc., employing connection protocols such as Serial Advanced Technology Attachment (SATA), Integrated Drive Electronics (IDE), IEEE-1394, Universal Serial Bus (USB), fiber channel, Small Computer Systems Interface (SCSI), etc. The memory drives may further include a drum, magnetic disc drive, magneto-optical drive, optical drive, Redundant Array of Independent Discs (RAID), solid-state memory devices, solid-state drives, etcetera.

Furthermore, memory storage device 506 may include one or more computer-readable storage media utilized in implementing embodiments consistent with the present disclosure. Generally, a computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., non-transitory. Examples include Random Access Memory (RAM), Read-Only Memory (ROM), volatile memory, non-volatile memory, hard drives, Compact Disc (CD) ROMs, Digital Video Disc (DVDs), flash drives, disks, and any other known physical storage media.

The memory storage device 506 may store a collection of program or database components, including, without limitation, an operating system 522, an application instruction 524, and a user interface element 526. In various embodiments, the operating system 522 may facilitate resource management and operation of the computer system 502. Examples of operating systems include, without limitation, APPLE® MACINTOSH® OS X®, UNIX®, UNIX-like system distributions (E.G., BERKELEY SOFTWARE DISTRIBUTION® (BSD), FREEBSD®, NETBSD®, OPENBSD, etc.), LINUX® DISTRIBUTIONS (E.G., RED HAT®, UBUNTU®, KUBUNTU®, etc.), IBM® OS/2®, MICROSOFT® WINDOWS® (XP®, VISTA®/7/8, 10 etc.), APPLE® IOS®, GOOGLE™ ANDROID™ BLACKBERRY® OS, or the like.

The application instructions 524 may include instructions that when executed by the processor 504 cause the processor 504 to perform one or more techniques, steps, procedures, and/or methods described herein, such to irrigate a site and irradiate a site as outlined herein. For example, application instructions 524, when executed by processor 504 can cause processor 504 to perform the method 400.

The user interface elements 526 may facilitate display, execution, interaction, manipulation, or operation of program components through textual or graphical facilities. For example, user interfaces may provide computer interaction interface elements on a display system operatively connected to the computer system 502, such as cursors, icons, checkboxes, menus, scrollers, windows, widgets, etcetera. The user interface elements 526 may be employed by application instructions 524 and/or operating system 522 to provide, for example, a user interface with which a user can interact with computer system 502. In some embodiments, the user interface elements 526 may be integrated with the display (e.g., display 124).

With respect to the use of substantially any plural and/or singular terms herein, those having skill in the art can translate from the plural to the singular and/or from the singular to the plural as is appropriate to the context and/or application. The various singular and/or plural permutations are expressly set forth herein for sake of clarity and not limitation.

It will be understood by those within the art that, in general, terms used herein, and are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended. For example, as an aid to understanding, the detail description may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to disclosures containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

All of the devices and/or methods disclosed and claimed herein can be made and executed without undue experimentation in light of the present disclosure. While the devices and methods of this disclosure have been described in terms of preferred embodiments, it may be apparent to those of skill in the art that variations can be applied to the devices and/or methods and in the steps or in the sequence of steps of the method described herein without departing from the concept, spirit, and scope of the disclosure. All such similar substitutes and modifications apparent to those skilled in the art are deemed to be within the spirit, scope and concept of the disclosure as defined by the appended claims.

What is claimed is:

1. A method of treating a target, comprising:
irrigating a treatment site with a bubble activation fluid, the bubble activation fluid comprising a liquid base and a compound dissolved in the liquid base; and
delivering, via an optical fiber coupled to a laser source, laser light,
wherein the laser light causes the liquid base of the bubble activation fluid to vaporize and the vapors to be trapped by the dissolved compounds to form a bubble in the treatment site, and
wherein the laser light is transmitted through the bubble to be incident on a target.

2. The method of claim 1, wherein the liquid base comprises distilled water and the compound comprises sodium chloride and wherein the bubble activation fluid is carbonated.

3. The method of claim 1, wherein the liquid base comprises distilled water and the compound comprises a surfactant, a biocompatible surfactant, a detergent, or a foaming agent.

4. The method of claim 1, wherein the liquid base comprises distilled water and the compound comprises a hydrophilic chain.

5. The method of claim 1, wherein the liquid base comprises distilled water and the compound comprises a an organic amphiphilic.

6. The method of claim 5, wherein the dissolved compounds form micelle structures to trap portions of the vaporized liquid base to reduce a surface tension of the bubble.

7. The method of claim 1, wherein the bubble activation fluid has a lower boiling point that saline solution, wherein the saline solution comprises 9 grams of sodium chloride dissolved in 1 liter of distilled water.

8. A system comprising:
a laser source arranged to deliver laser light to a treatment site via an optical fiber;
a fluid reservoir comprising a bubble activation fluid, the bubble activation fluid comprising a liquid base and a compound dissolved in the liquid base; and
a pump coupled to the fluid reservoir, the pump rearranged to irrigate the treatment site with the bubble activation fluid,
wherein the laser light causes the liquid base of the bubble activation fluid to vaporize and the vapors to be trapped by the dissolved compounds to form a bubble in the treatment site, and
wherein the laser light is transmitted through the bubble to be incident on a target.

9. The system of claim 8, wherein the liquid base comprises distilled water and the compound comprises sodium chloride and wherein the bubble activation fluid is carbonated.

10. The system of claim 8, wherein the liquid base comprises distilled water and the compound comprises a surfactant, a biocompatible surfactant, a detergent, or a foaming agent.

11. The system of claim 8, wherein the liquid base comprises distilled water and the compound comprises a hydrophilic chain.

12. The system of claim 8, wherein the liquid base comprises distilled water and the compound comprises a an organic amphiphilic.

13. The system of claim 8, wherein the dissolved compounds form micelle structures to trap portions of the vaporized liquid base to reduce a surface tension of the bubble.

14. The system of claim 8, wherein the bubble activation fluid has a lower boiling point that saline solution, wherein the saline solution comprises 9 grams of sodium chloride dissolved in 1 liter of distilled water.

15. The system of claim 8, comprising the optical fiber.

16. The system of claim 8, wherein the laser source comprises a Thulium fiber laser (TFL) or a Holmium:YAG laser.

17. A fluid comprising:
a liquid base; and
a compound dissolved in the liquid base,
wherein the liquid base is arranged to vaporize responsive to laser light, and
wherein the dissolved compounds are arranged to trap portions of the vaporized base to form a bubble in the liquid base.

18. The fluid of claim 17, wherein the liquid base comprises distilled water and the compound comprises sodium chloride and wherein the bubble activation fluid is carbonated.

19. The fluid of claim 17, wherein the liquid base comprises distilled water and the compound comprises a surfactant, a biocompatible surfactant, a detergent, or a foaming agent.

20. The fluid of claim 17, wherein the bubble activation fluid has a lower boiling point that saline solution, wherein the saline solution comprises 9 grams of sodium chloride dissolved in 1 liter of distilled water.
